(19) United States
(12) Patent Application Publication
(10) Pub. No.: US 2007/0134298 A1
(43) Pub. Date: Jun. 14, 2007

(54) DIETARY SUPPLEMENT

(75) Inventor: Jerzy A. Georgiades, Houston, TX (US)

Correspondence Address: RANKIN, HILL, PORTER & CLARK, LLP, 925 EUCLID AVENUE, SUITE 700, CLEVELAND, OH, 44115-1405, US

(73) Assignee: REGEN BIOTECH LIMITED, London (GB)

(21) Appl. No.: 11/626,696

(22) Filed: Jan. 24, 2007

Related U.S. Application Data
(63) Continuation of application No. 10/932,535, filed on Sep. 2, 2004, now abandoned.
Application No. 10/932,535 is a continuation of application No. 09/719,944, filed on May 2, 2001, now abandoned.

(30) Foreign Application Priority Data
Jun. 16, 1998 (GB) 9813031.3

(51) Int. Cl. A61K 38/21; A61K 47/00; A61K 9/68; A61K 33/04; A61K 35/20

(52) U.S. Cl. 424/439; 424/440; 424/85.1; 424/85.2; 424/85.7; 424/85.6; 424/702; 424/535

(57) ABSTRACT

A dietary supplement comprises a combination of colostrinin and at least one of lactoferrin and selenium.

### CROSS-REFERENCE TO RELATED APPLICATIONS

This application is a continuation of co-pending application Ser. No. 10/932,535, filed Sep. 2, 2004, which is a continuation of application Ser. No. 09/719,944, filed May 2, 2001, which is a 371 of PCT/GB99/01878, filed Jun. 15, 1999, and claims priority to GB9813031.8 filed Jun. 16, 1998.

The present invention relates to a dietary supplement and, in particular, to a dietary supplement for promoting the functioning of the immune system. The invention also relates to baby formulas.

When a baby is born, its immune system is normally dormant and non-functioning but, as the baby grows, the immune system becomes active. There has recently been a hypothesis that mother's colostrum contains components which contribute to the awakening and development of the immune system. One particular such component is called colostrinin. It is found, inter alia, in ovine and human colostrum.

As a result of our studies, we have found that the administration of colostrinin to an infant may be of singular importance to the full development of the immune system, and that it is possible that an infant fed solely on bottle formula milk preparations may, as a result of not receiving colostrinin, have a poorly developed immune system. An imperfectly developed immune system can lead to the development of serious diseases such as atopic allergies including, for example, as asthma and skin allergies. There have even been reports that a reduced function of the immune system can lead to senility in old age and, possibly, to Aizheimer's disease.

It is impractical to solve this problem by trying to take steps to ensure that all infants are breast fed, because some mothers are physically unable to breastfeed, and others may not be able to breastfeed because they are undergoing treatment themselves and are taking drugs which should not be passed on to the baby through breast milk. Also, in some areas of the world, there is a social stigma attached to breastfeeding.

We have now devised a dietary supplement formula for promoting the correct functioning of the immune system. The supplement can be given to non-breast fed infants, for example by inclusion in their baby formulas or powdered milk feed. It can also be given to breast-fed infants and to children and adults at any time of their life, especially if they show signs of immune deficiency. Thus, the invention provides a way of treating an individual with a view to promoting their immune system whether or not they have been breast-fed, and whatever their state of health.

The dietary supplement of the present invention comprises colostrinin in combination with at least lactoferrin. We have found that this combination of substances exhibits synergism.

By “dietary supplement” we mean a preparation or formulation which is added to or otherwise included in a person's normal diet, and is present in addition to the normal diet. Thus, for example, a dietary supplement of the invention can be:
(a) in the form of a liquid or solid, e.g., powder or as individual dosage units such as baby food formula, tablets or the like to be added to food or drinks, or taken with them,
(b) added to a foodstuff during its preparation, such as added to powdered milk feed for babies otherwise included in children's and adults' foodstuffs.

By “dietary supplement” we do not intend to embrace foodstuffs per se that may naturally contain the components of the supplement according to the invention.

The synergism can be further enhanced by the addition of selenium to the composition.

The lactoferrin, selenium and colostrinin present in the preferred food supplement of the invention can each be of natural or synthetic origin, e.g., produced by recombinant DNA technology. The supplements will normally also include a physiologically acceptable diluent or carrier such as is appropriate to the particular use intended.

In a preferred embodiment, the selenium is in the form of a physiologically acceptable selenoprotein, such as selenocysteine. The selenium can be provided in the form of glutathione peroxidase. The selenium can be provided in the form a complex in which it is bound to Lactobacillus acidophilus or yeast protein. Furthermore, the selenium protein complex is preferably human and may be from a recombinant or natural source. Selenium is known to be a weak inducer of the cytokines and in particular of gamma interferon. It is particularly preferred that the selenium be present in the dietary supplement in the form of selenium rich proteins rather than as a salt, since when administered as for example selenium picollinate, it is generally not fully utilized by the body.

The term “colostrinin”, as used herein refers to a complex of polypeptides which, in its natural form, is obtained from any mammalian colostrum. Colostrum is the thick, yellowish fluid produced by a mammalian mother's breasts during the first few days after childbirth. It is the first lacteal secretion post parturition and it contains a high concentration of immunoglobulins (IgG, IgM and IgA) and nonspecific proteins. It is replaced by mature breast milk about four to five days after birth. Compared with mature breast milk, colostrum contains low sugar and iron. However, colostrum is richer in lipids, proteins, mineral salts, vitamins and immunoglobulins. It also contains various floating cells such as granular and stromal cells, neutrophils, monocytelmacrophages and lymphocytes and includes growth factors, hormones and cytokines.

Various factors have been isolated and characterized from mammalian colostrum. In 1974, Janusz et al (FEBS Lett., 49, 276-279) isolated a proline-rich polypeptide (PRP) from ovine colostrum. It has since been discovered that mammals other than sheep have analogues of PRP as a component of their colostrum. PRP has since been called colostrinin (and is sometimes called colostrinine).

M. Janusz & J. Lisowski in “Proline-Rich Polypeptide (PRP)—an Immunomodulatory Peptide from Ovine Colostrum” (Archivum Immunologiae et Therapiae Experimentalis, 1993, 41, 275-279) mentioned that PRP from ovine colostrum has immunotropic activity in mice.

A. Dubowska-Inglot et al in “Colostrinine: a proline-rich polypeptide from ovine colostrum is a modest cytokine inducer in human leukocytes” (Archivum Immunoiogiae et Therapiae Experimentaiis, 1996, 44, 215-224) discussed the use of colostrinin in the treatment of Alzheimer's disease. The use of colostrinin in the treatment of Alzheimer's disease, and other conditions, was also discussed in WO-A-98/14473.

Colostrinin, in its natural form, is obtained from mammalian colostrum. As described in WO-A-98/14473,analysis by electrophoresis and chromatography has shown that colostrinin has the following properties:
(i) it has a molecular weight in the range 16,000 to 26,000 Daltons (this was shown by electrophoresis in the presence of SDS);
(ii) it is a dimer or trimer of sub-units each sub-unit having a molecular weight in the range 5,000 to 10,000 Daltons (this was shown by acrylamide gel electrophoresis in the presence of SDS);
(iii) it contains proline, and the amount of proline is greater than the amount of any other single amino acid (this can be shown by conventional amino acid analysis).

It has also been shown that colostrinin and the sub-units making up the colostrinin are non-polar.

By means of these techniques it was shown that ovine colostrinin has a molecular weight of about 18,000 Daltons, is made up of three non-covalently linked sub-units each having a molecular weight of about 6,000 Daltons and includes about 22 wt % proline. The amino-acid composition of ovine colostrinin was shown to be made up of the following number of residues per sub-unit-lysine-2, histidine-1, arginine-0, aspartic acid-2, threonine-4, serine-3, glutamic acid-6, proline-11, glycine-2, alanine-0, valine-5, methionine-2, isoleucine-2, leucine-6, tyrosine-1, phenylalanine-3 and cysteine-0.

The colostrinin used in the food supplement of the invention may be derived naturally from any mammalian source, such as humans, bovine, goats or sheep. Alternatively, the colostrinin may be made synthetically, for example, by recombinant DNA techniques. The colostrinin need not necessarily be in a pure form but may instead be, for example, partially purified as, for example, I_{g}G-colostrinin complex, or in a crude preparation form like whey, so long as the form is physiologically acceptable.

The source of the lactoferrin is also not critical but it should preferably be of bovine, ovine or human origin (or derived therefrom). Most preferably, human lactoferrin and/or human recombinant lactoferrin are used.

The preferred amount of each ingredient per unit dose of the dietary supplement is as follows: colostrinin from about 12½ micrograms to about 200 micrograms; lactoferrin from about 10 micrograms to about 100 milligrams; and selenium, in the form of seleno-cysteine, from about 2.5 to about 100 micrograms. However, for young babies the preferred amount is below 2.5 micrograms, for example 0 to 1.0 microgram.

The preferred dosage of the dietary supplement of the invention is one preferred unit dose per day.

The dietary supplement of the invention may further include other biologically active substances such as the cytokines present in colostrum other than colostrinin, and hormones. For example, the supplement may include a natural cytokine preparation containing members of the interferon family (including interferon α and interferon γ), interleukin 1-α, interleukin 1-3,interleukin-6, 8, 10, 12, 16,tissue necrosis factor α, G-CSF (granulocyte colony stimulating factor), M-CSF (macrophage CSF), TGFα (transforming growth factor) and TGFβ.

The physiologically acceptable carrier of the dietary supplement of the present invention is chosen to be suitable for the intended use. Examples of suitable carriers include for example a solution of the hydrolysates of β casein in the form of 6.000 m.w. peptides, phosphate buffered saline (PBS), and whey.

The most preferred route for administering a dietary supplement of the invention is oral, especially in a form in which the supplement is maintained in contact with the oral and/or pharyngeal and/or intestinal tract mucosa. One preferred form is that of a baby food formula. Another preferred form is that of a lozenge, designed to be dissolved in the mouth. In the lozenge or other form, the dietary supplement may further include various flavoring or sweetening agents such as sucrose, mannose, lactose, maltose, trehalose, cold water soluble starch or other such ingredients known in the art.

As will be understood, the food supplement of the invention can be in a number of other forms such as powders, tablets, or liquid drinks and baby formulas. When in powder form, they can be added to a foodstuff such as, for example, a powdered milk formulation (or cheese or yoghourt or indeed any other foodstuff). The source of the milk is not important and may, for example, be cow, goat or sheep. The powdered milk formulation may be made up with a liquid to form a drink.

In another form, the dietary supplements of the invention can be included in a cheese. The source of milk forming the base of the composition to form the cheese is not important, but may include cow, goat or sheep.

The dietary supplement of the invention can also be added to the whey of goat, cow or sheep milk origin which whey may be obtained during cheese production. The whey product containing the dietary supplement may be consumed as a drink.

In a further aspect of the present invention, there is provided the use of colostrinin in combination with lactoferrin in the manufacture of a medicament for bringing about an improvement in an individual's immune system.

The dietary supplement of the present invention can result, in adults, in an increase in energy and an apparent increase in clarity of thinking.

The dietary supplement of the invention should preferably not be used for more than 21 days continuously. This is because the phenomenon of tachyphylaxis may otherwise be induced. Tachyphylaxis is the gradual loss of an individual's capability to synthesize cytokines. In this situation an adverse reaction may be experienced. Induction of tachyphylaxis may be avoided by discontinuing the use of the dietary supplement of the invention after 21 days for a period of not less than 3 weeks. Following this brief pause, a new cycle of use can be initiated.

According to another aspect of the present invention there is provided a method of stimulating an individual's immune system, which method consists essentially of administering a dietary supplement of the present invention in unit dosage form, preferably each day for 21 consecutive days.

The invention described above relates to a dietary supplement containing colostrinin and lactoferrin, and to certain uses thereof. In another aspect the invention relates to a dietary supplement comprising colostrinin and selenium; this dietary supplement may be used in the same way as the dietary supplement described above, and may have the same additional components. In yet another aspect the invention relates to a dietary supplement comprising colostrinin and at least one of the cytokines listed above; this dietary supplement may be used in the same way as the dietary supplement described above, and may have the same additional components.

In order that the invention may be more fully understood, the following Examples are given by way of illustration only.

### EXAMPLE 1

Lozenge Formulation

The composition of a lozenge formulation of an example of the dietary supplement of the invention per unit dose is as follows:

| Inqredient | Amount | |
|---|---|---|
| Sucrose, lactose or | 25 | mg |
| trehalose and/or | | |
| Cold-water-soluble starch | 42 | mg |
| Phosphate Buffered Saline | (if required) | |
| Natural colostrinin | 100 | μg |
| Selenium (metalloprotein) | 5 | μg |
| or other seleno-cysteine- | | |
| containing proteins | | |
| Purified recombinant | 10 | mg |
| human lactoferrin | | |

In all these examples, the colostrinin can be obtained by processes well known in the art. Such processes are described, for example, in the references discussed above. The other materials are also readily available.

### EXAMPLE 2

Method of manufacture of lozenqe formulation

A starch gel-based lozenge containing colostrinin, lactoferrin and selenium is prepared by combining 150 g sucrose, 550 ml phosphate 0.15 mm buffered saline, and 250 g of cold-water-soluble starch such as that described in U.S. Pat. No. 4,465,702, heating the mixture with stirring to a temperature of 75° C., cooling the mixture to 30° C. and thereafter blending into the paste-like mass with 50 ml PBS containing 3 mg purified human colostrinin, 4.5 mg selenium (rich protein) and 300 mg purified recombinant human lactoferrin. The mixture is then formed into multiple portions of 5 to 10 grams each, which set upon standing under drying conditions to a starch candy gel-like consistency. The lozenges thereby produced can be administered to a patient singly or in combination. The patient is instructed to hold the lozenge in his mouth until it is completely dissolved to release the components for contact with the oral mucosa.

### EXAMPLE 3

Powdered Milk Formulation

A formulation for feeding to a baby post weaning from mother's milk, is:

| Proprietary milk powder | 4 | g** |
|---|---|---|
| (e.g. SMA ™, WHITE ™ by Sma | | |
| Nutrition, Maidenhead, U.K.*) | | |
| Natural ovine colostrinin | 150 | μg |
| Selenium (bound to | 8.25 | μg (free selenium) |
| lactobacillus acidophilus) | | |
| Human Recombinant Lactoferrin | 1.0 | mg |
| *SMA ™ ingredients quoted as lactose, skimmed milk powder, vegetable oils, emulsifier (soya lecithin), potassium bicarbonate, vitamin C, taurine, ferrous sulphate, zinc sulphate, cytidine-5′-monophosphate, disodium uridine-5′-monophosphate, vitamin E, adenosine-5′-monophosphate, niacin, disodium inosine-5′-monophosphate, disodium guanosine-5′-monophosphate, pantothenic acid, vitamin A, copper sulphate, thiamin, | | |
| # vitamin B, riboflavin, beta-carotene, manganese sulphate, foiic acid, vitamin K, potassium iodide, biotin, vitamin D, vitamin B. Although the manufacturer lists ferrous sulphate as an ingredient, we prefer not to include this material or any other iron containing compounds. | | |

**Follow manufacturer's instructions for dosage guide e.g. weight 6.5 kg, approximate age of baby 4 months, 7 level scoops in 200 mi cooled (freshly boiled) water.

### EXAMPLE 4

Baby Food Formulas

The following formulations may be used for very young babies:

Formulas for new born 1-7 days old:

| | |
|---|---|
| Natural Colostrinin | 50 μg per serving. |
| (antibody - colostrinin complex) | |
| Lactoferrin | 100 μg per serving. |
| (human recombinant or natural bovine) | |

Formulas for 8-14 days old babies:

| | |
|---|---|
| Natural colostrinin | 5.0 μg per serving. |
| Lactoferrin | 100 μg per serving. |
| Selenium in form of seleno-cysteine | 1.0 μg per serving. |

Formulas for 15-30 days old babies.

| | |
|---|---|
| Natural colostrinin | None |
| Lactoferrin | 50 μg per serving. |
| Selenium | 0.5 μg per serving. |

Formulas for 31-45 days old babies.

| | |
|---|---|
| Natural colostrinin complex | 10 μg per serving. |
| Lactoferrin | 50 μg per serving. |
| Selenium | 0.5 μg per serving. |

It will be appreciated that modifications may be made to the invention described above.

What is claimed is:

1. A dietary supplement comprising colostrinin in combination with lactoferrin.

2. A dietary supplement according to claim 1, further comprising selenium.

3. A dietary supplement according to claim 2, wherein the selenium is in the form of a physiologically acceptable selenoprotein.

4. A dietary supplement according to claim 1, further comprising at least one cytokine selected from interferon α, interferon γ, interleukin 1- α, interleukin 1-3, interleukin 6, 8, 10, 12, 16,tissue necrosis factor α, G-CSF, M-CSF, TGF a and TGFβ.

5. A dietary supplement comprising colostrinin in combination with selenium.

6. A dietary supplement comprising colostrinin in combination with at least one cytokine selected from interferon α, interferon γ, interleukin 1-α, interleukin 1-3, interleukin 6, 8, 10, 12, 16,tissue necrosis factor α, G-CSF, M-CSF, TGFA and TGFβ.

7. A baby food formula comprising a dietary supplement according to claim 1.

8. The use of dietary supplement according to claim 1 in a baby food formula.

9. A tablet, lozenge or other solid oral dosage form comprising 12.5 micrograms to 200 micrograms colostrinin, 10 micrograms to 100 milligrams lactoferrin, and 2.5 to 100 micrograms seleno-cysteine in combination with a physiologically acceptable carrier.

10. The use of colostrinin in combination with lactoferrin in the manufacture of a medicament for improving the immune system of mammals.

11. The use of colostrinin in combination with selenium in the manufacture of a medicament for improving the immune system of mammals.

12. The use of colostrinin in combination with at least one cytokine selected from interferon α, interferon γ, interleukin 1-α, interleukin 1-3,interleukin 6, 8, 10, 12, 16, tissue necrosis factor α, G-CSF, M-CSF, TGFA and TGFβ in the manufacture of a medicament for improving the immune system of mammals.

13. A method of stimulating the immune system of a mammal, comprising administering a dietary supplement according to any one of claim 1 in unit dosage form.

14. A method according to claim 13, wherein the unit dosage form comprises 12.5 micrograms to 200 micrograms colostrinin, 10 micrograms to 100 milligrams lactoferrin, and 2.5 to 100 micrograms seleno-cysteine in combination with a physiologically acceptable carrier.

15. A method according to claim 13, wherein one unit dose of the dietary supplement is administered each day for a first period of not more than three weeks, then no dosage is administered for a subsequent period of up to three weeks.

16. A baby food formula comprising a dietary supplement according to claim 5.

17. The use of a dietary supplement according to claim 5 in a baby food formula.

18. A method of stimulating the immune system of a mammal, comprising administering a dietary supplement according to claim 5 in unit dosage form.

19. A method according to claim 14, wherein one unit dose of the dietary supplement is administered each day for a first period of not more than three weeks, then no dosage is administered for a subsequent period of up to three weeks.
